(19) United States
(12) Patent Application Publication
(10) Pub. No.: US 2022/0401109 A1
(43) Pub. Date: Dec. 22, 2022

(54) SYSTEMS AND METHODS FOR TREATING THE LEFT ATRIAL APPENDAGE

(71) Applicant: Ventrimend, Inc, Lake Forest, CA (US)

(72) Inventors: David Zarbatany, Lake Forest, CA (US); Ricardo Roman, Lake Forest, CA (US)

(73) Assignee: Ventrimend, Inc, Lake Forest, CA (US)

(21) Appl. No.: 17/846,369

(22) Filed: Jun. 22, 2022

Related U.S. Application Data
(60) Provisional application No. 63/213,620, filed on Jun. 22, 2021.

(51) Int. Cl. A61B 17/12

(52) U.S. Cl. CPC A61B 17/12122; A61B 17/12172; A61B 2017/12054

(57) ABSTRACT

The present invention describes systems and methods for treating the left atrial appendage with an implant to fluidly seal the left atrial appendage and prevent blood from flowing from the left atrial appendage to the left atrium. The closure implant includes a braided disk, a proximal petal anchor and a distal petal anchor positioned on an implant shaft. The braided disk includes two diameters: a proximal diameter sized to engage a wall area in the left atrium around the LAA ostium; and a distal diameter sized to fit within the LAA ostium. The proximal and distal petal anchors include asymmetrical petals arranged like the petals of a flower configured to engage the wall of the LAA to anchor the implant. The implant is designed to be delivered to the heart using a catheter-based delivery system.

### CROSS-REFERENCE TO RELATED APPLICATIONS

This application claims priority to U.S. Provisional Patent Application No. 63/213,620, filed Jun. 22, 2021, the entire disclosure of which is incorporated by reference herein.

### FIELD

The present invention relates generally to the field of surgery, and more specifically, to treatment of the left atrial appendage.

### BACKGROUND

The left atrial appendage (“LAA”) is a cavity extending from the lateral wall of the left atrium between the mitral valve and the root of the left pulmonary veins. The LAA normally contracts with the rest of the left atrium during a normal heart cycle, keeping blood from becoming stagnant therein, but often fails to contract with any vigor in patients experiencing atrial fibrillation (“AF”) due to the coordinate electrical signals associated with AF, in patients with AF and other abnormal heart conduction. The result is that blood tends to pool in the LAA, which can lead to the formation of blood clots therein. The blood clots can then propagate out from the LAA into the left atrium. Since blood from the left atrium and ventricle supply the heart and brain, blood clots from the LAA can obstruct blood flow thereto, causing heart attacks, strokes, or other organ ischemia. Blood clots form in the LAA in about 90% of patients with atrial thrombus. Patients with AF account for one of every six stroke patients, and thromboemboli originating from the LAA are the suspected culprit in the vast majority of these cases. More than 3 million Americans have AF, which increases their risk of stroke by a factor of 5. Elimination or containment of thrombus formed within the LAA of patients with AF will significantly reduce the incidence of stroke in those patients.

The left atrial appendage (LAA) has been identified as an important source of AF triggers, particularly among patients with structural heart disease, nonparoxysmal AF, and AF recurrence after AF ablation. LAA electrical isolation (LAAEI) has been viable adjunctive strategy to treating patients with AF in addition to PV isolation. LAAEI is an adjunctive strategy to PV isolation for maintenance of SR. Mechanical force displaced radially at the ostium of the LAA will create electrical isolation by compressing the myocyte cells at the contact site and inhibit the exchange of sodium and calcium, thus elimination of the refractory process of cardiac myocytes. The resulting cellular response causes apoptosis or programmed cellular death. This process decouples active cells causing electrically deactivated cells and produces a focal line of non-conductive tissue, ultimately causing tissue necrosis electrically disassociating the LA from LAA tissue.

In AF, the LAA can cause a significant amount of arrhythmogenic sources (ectopic activity, PV-like potentials) which is an important initiating source of AF. In patients with previous ablation procedures, the LAA can continue to initiate and/or maintain the AF arrhythmia. LAA electrical isolation in addition to standard ablation will have an incremental benefit to achieve freedom from ALL atrial arrhythmias in patients with atrial fibrillation. The LAA has limited contractibility when in AF and if it is isolated it would have no contractibility. LAA electrical isolation with a LAA polymer filling the LAA would ensure patient safety and improve AF outcomes while reducing stroke.

Percutaneous LAA occlusion has been demonstrated to be as effective as anticoagulation drugs in reducing the risk of thromboembolic stroke in patients with AF. LAA occlusion is an elegant method of improving success rates of ablation for AF whilst also mitigating stroke risk and reducing the bleeding risks from long-term anticoagulation.

Accordingly, there remains a need for systems and methods that provide solutions to the problems of current systems. The present invention is directed toward meeting these needs.

### SUMMARY

The present invention describes systems and methods for treating the left atrial appendage (LAA) with an implant to fluidly seal the LAA and prevent blood from flowing from the LAA to the left atrium. The closure implant includes a braided disk, a proximal petal anchor and a distal petal anchor positioned on an implant shaft. The braided disk may be self-expanding and have two diameters: a proximal diameter sized to engage a wall area in the left atrium around the LAA ostium; and a distal diameter sized to fit within the LAA ostium. The proximal and distal petal anchors include asymmetrical petals having a rearward curvature arranged like the petals of a flower configured to engage the wall of the LAA to anchor the implant. The implant is designed to be delivered to the heart using a catheter-based delivery system.

### BRIEF DESCRIPTION OF THE DRAWINGS

FIG. 1 is a sectional view of the heart anatomy.

FIG. 2 shows one embodiment of an implant for the LAA having an expandable braided disk coupled to a proximal petal anchor and a distal petal anchor.

FIG. 3 shows the implant coupled to a delivery system.

FIG. 4 is a front view showing the asymmetrical petals of the proximal and distal pedal anchors.

FIG. 5 shows one example of the placement of the implant in the LAA.

FIG. 6 shows delivery of the implant through the vascular system and the heart 10 to the LAA.

FIGS. 7A-7F show delivery of an implant to the LAA through a delivery system.

FIG. 8 is a perspective showing the components of the connect/disconnect feature.

FIG. 9 is perspective view showing the implant coupled to the delivery system.

FIG. 10 is perspective view of the connect/disconnect feature of the invention showing the delivery system coupled to the implant.

FIG. 11 is a sectional view showing the engagement of the distal end of the shaft and engagement arms coupled to the coupler and slots.

FIG. 12 is a sectional view showing the delivery system disengaged from the implant.

### DETAILED DESCRIPTION

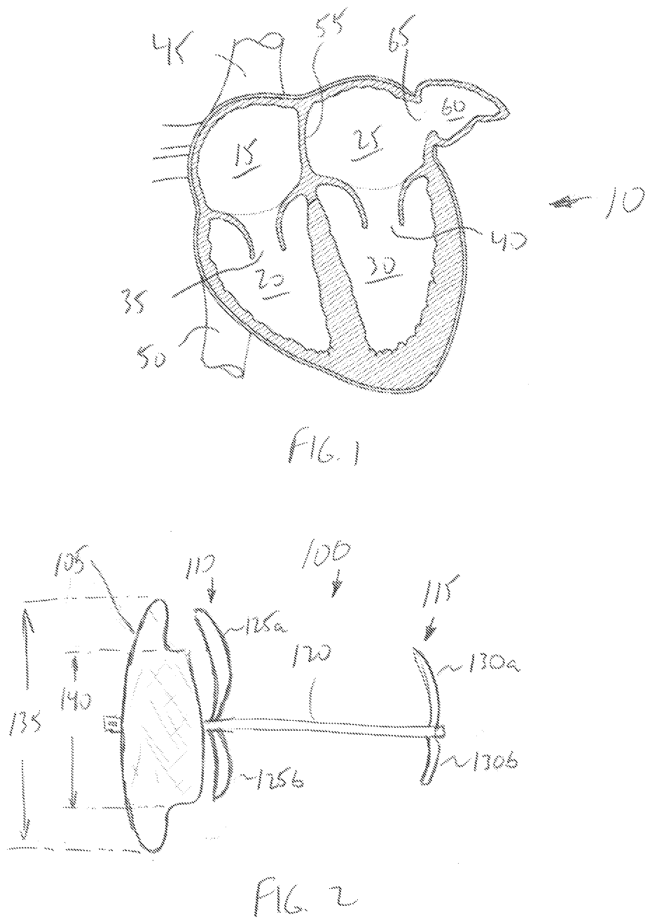

FIG. 1 is a sectional view showing the anatomy of a heart 10. The heart 10 includes four chambers: a right atrium 15, a right ventricle 20, a left atrium 25 and a left ventricle 30. A tricuspid valve 35 allows blood to flow from the right atrium 15 into the right ventricle 20. Blood enters the right atrium 15 from the superior vena cava 45 and the inferior vena cava 50 blood vessels. The blood flows into the right atrium 15 and then through the tricuspid valve 35 into the right ventricle 20. Blood flows from the right ventricle 20 into the pulmonary aorta to the lungs. Once through the lungs, the blood flows back to the heart 10 and into the left atrium 25. The blood from the left atrium 25 flows through the mitral valve 40 into the left ventricle 30 and out of the heart 10 to the ascending aorta. The right atrium 15 and left atrium 25 are separated by the atrial septum 55.

Extending from left atrium 25 is the left atrial appendage (LAA) 60, a cavity structure that normally contracts with the left atrium 25 when the heart pumps. Sometimes, the LAA 60 fails to contract correctly and the result is that blood tends to pool in the LAA 60, which can lead to the formation of blood clots within the LAA 60. The blood clots can then propagate out from the LAA 60 through the LAA ostium 65 into the left atrium 25. Since blood from the left atrium 25 and left ventricle 30 supply the heart and brain, blood clots from the LAA 60 can obstruct blood flow thereto, causing heart attacks, strokes, or other organ ischemia.

FIG. 2 shows one embodiment of an implant 100 designed to seal the LAA and prevent clots from forming and/or entering the left atrium 25 from the LAA 60. The implant 100 is designed to be inserted into the LAA 60 and fluidly seal the LAA ostium 65. The implant 100 can be implanted in a surgical procedure or via one or more catheter-based delivery systems in an interventional procedure.

The implant 100 includes a braided disk 105, a proximal petal anchor 110 and a distal petal anchor 115 positioned on an implant body or shaft 120. The braided disk 105 includes a covering material configured to fluidly seal the LAA 60 from the left atrium 25 to reduce risk of clot formation and migration that might otherwise result (e.g., in a patient with AF or otherwise compromised LA function).

In the embodiment shown, the braided disk 105 includes two diameters: a proximal diameter 135 sized to engage a wall area around the LAA ostium 65 in the left atrium 25; and a distal diameter 140 sized to fit within the LAA ostium 65. The distal diameter 140 may keep the braided disk 105 centered in the LAA ostium 65. The proximal and distal diameters 135, 140 may be adjustable diameters that are configured to expand to fit different size LAA ostium 65 openings or shapes. In other embodiments, the braided disk 105 may have more than two diameters. In some embodiments, the expandable braided disk 105 may further be configured to impart a force circumferentially about the LAA ostium 65 to disrupt cell to cell conduction within the tissue and electrically isolate the LAA 60.

The braided disk 105 includes an expandable mesh structure covered with a covering material to form a fabric seal. The braided disk 105 is made from a shaped memory mesh, such as nitinol, that is configured to self-expand after being compressed. The expandable braided disk 105 may be made of a nitinol wire mesh. The covering material may be a biocompatible material. In some embodiments, the covering comprises a material selected from the group consisting of: a woven material; a fabric; a wire mesh; polyethylene terephthalate; a sponge; cellulose; synthetic fiber; cotton; rayon; hydrogel; a coagulant; a biodegradable material; a non-biodegradable material and combinations of one, two, or more of these.

In the embodiment shown, the proximal petal anchor 110 includes asymmetrical petals 125a, 125b and the distal petal anchor 115 includes asymmetrical petals 130a, 130b arranged like the petals of a flower configured to engage the wall of the LAA 60 to anchor the implant 100. The petals are self-expanding and are made of a shape-memory wire, such as a nitinol wire, that may be pre-shaped in a petal shape to allow the petals to be delivered to the LAA in a collapsed or compressed shape within a delivery system. Then once delivered, shape-memory wire self-expands the petal back into the pre-shaped petal when released from the delivery system in the LAA.

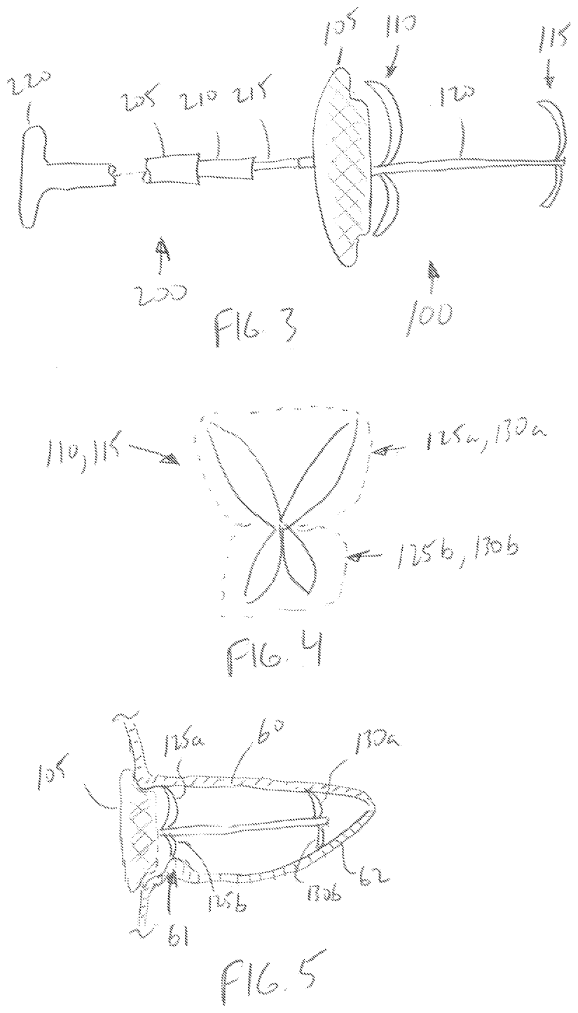

FIG. 3 shows the implant 100 coupled to the distal end of a delivery system 200. The implant 100 can be delivered to the LAA 60 in a surgical procedure or via the catheter-based delivery system 200 in an interventional procedure. The delivery system 200 may include:
Steerable Introducer Sheath with Dilator 205
   Short Steering section for up to 30-degree curve
   PTFE Lined
   Polyethylene dilator
Inner Steerable Sheath 210
   180-degree steering section
   PTFE Lined
Therapy “depth” Catheter 215
   Braided, multi-durometer, no ptfe shaft
   Connect/disconnect feature
Handle 220

The steerable introducer sheath with dilator includes an internal lumen that is configured and dimensioned to slidably receive the inner steerable sheath. The inner steerable sheath includes an internal lumen that is configured and dimensioned to slidably receive the catheter. The catheter is releasably coupled to the implant 100 at the connect/disconnect feature to deliver the implant 100 to the LAA 60. Once the implant 100 is deployed in the LAA 60, the connect/disconnect feature is disconnected and withdrawn.

FIG. 4 is a front view showing the asymmetrical petals of the proximal and distal pedal anchors 110, 115. In the embodiment shown, the proximal petal anchor 110 includes two petal groups 125a, 125b having two petals, and the distal petal anchor 115 includes two petal groups 130a, 130b having two petals. The petals have a rearward curvature, like a fishhook, configured to engage the walls of the LAA 60 with enough compressive force to anchor the implant 100 within the LAA 60 and resist pull-out. In other embodiments, the proximal and distal petal anchors 110, 115 may have more than two petal groups and/or more individual petals in each petal group. In other embodiments, there also may be more than proximal and distal petal anchors 110, 115, such as an intermediate petal anchor positioned between than the proximal petal anchor 110 and the distal petal anchor 115. This may depend on the length and/or size of the LAA 60.

As discussed above, the petals are made from a wire of shape-memory material that is configured to form the shape of the petal segment. The shape-memory material allows the petals to be collapsed or compressed for delivery to the LAA and then self-expand once in position. The petal wires may extend proximally and be manipulated to change the size and/or shape of the petal. Each petal wire may be manipulated separately to change size and/or shape, or the petals in each petal group may be linked to manipulated all the petals in the petal group at the same time. The adjustable petal wires for each petal allows adjustability of the petals to accommodate the anatomy or placement requirements of the implant. For example, the size of the petal may be changed to keep the implant in the center of the LAA, or may be changed to engage defects in the LAA, such as a bump in the wall.

FIG. 5 shows one example of the placement of the proximal petal anchor 110 implanted near a bump 61 in the proximal end of the LAA 60 and the distal petal anchor 115 positioned near the distal end of the LAA 60 where the lower side 62 of the LAA is at a steeper angle than the upper side. During delivery, the distal petal anchor 115 is expanded and rotated or torqued so that smaller petals 130b contact the steeper lower side 62 and the larger petals 130a contact the upper side to keep the implant 100 centered in the LAA 60. The proximal petal anchor 110 is then expanded and rotated or torqued so that smaller petals 125b contact the lower LAA bump 61 and the larger petals 125a contact the upper part of the LAA to keep the implant 100 centered in the LAA 60. The braided disk 105 is then expanded with the smaller diameter 140 positioned in the LAA ostium 65 and the larger diameter 135 engaging the wall area in the left atrium 25 around the LAA ostium 65.

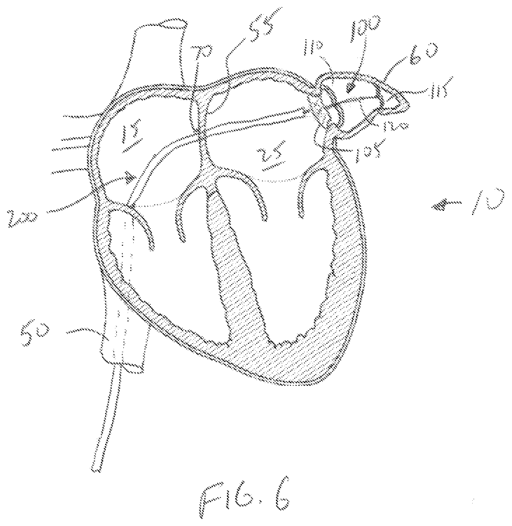

FIG. 6 shows delivery of the implant 100 through the vascular system and the heart 10 to the LAA 60. The delivery system 200 is advanced through the vascular system to the heart and into the right atrium 15. The delivery system 200 then goes through the atrial septum 55 to left atrium 25. This can be done by puncturing a hole 70 the wall of the atrial septum 55 between the right atrium 15 and the left atrium 25, or through a hole 70 of a patent foramen ovale or atrial septal defect, if present. Once in the left atrium 25, the distal end of the delivery system 200 is advanced to the LAA 60. The implant 100 is then advanced through a lumen in the delivery system 200 with the braided disk 105, proximal petal anchor 110, and the distal petal anchor 115 collapsed or compressed into a delivery configuration. The implant 100 exits the distal end of the lumen in the LAA 60 and self-expands with the proximal and distal petal anchors 110, 115 self-expanding and engaging the interior wall of the LAA 60, and the braided disk 105 self-expanding to contact the wall around the LAA ostium 56 to fluidly seal the LAA 60. The implant 100 is then disconnected from the delivery system 200 and the delivery system 200 is withdrawn. If the hole 70 in the septum 55 needs to be repaired, a closure implant may in used to close the hole 70. To deliver the closure implant, the distal end of the delivery system in positioned proximate the hole 70 and the closure implant is delivered through the distal of the lumen to seal the hole.

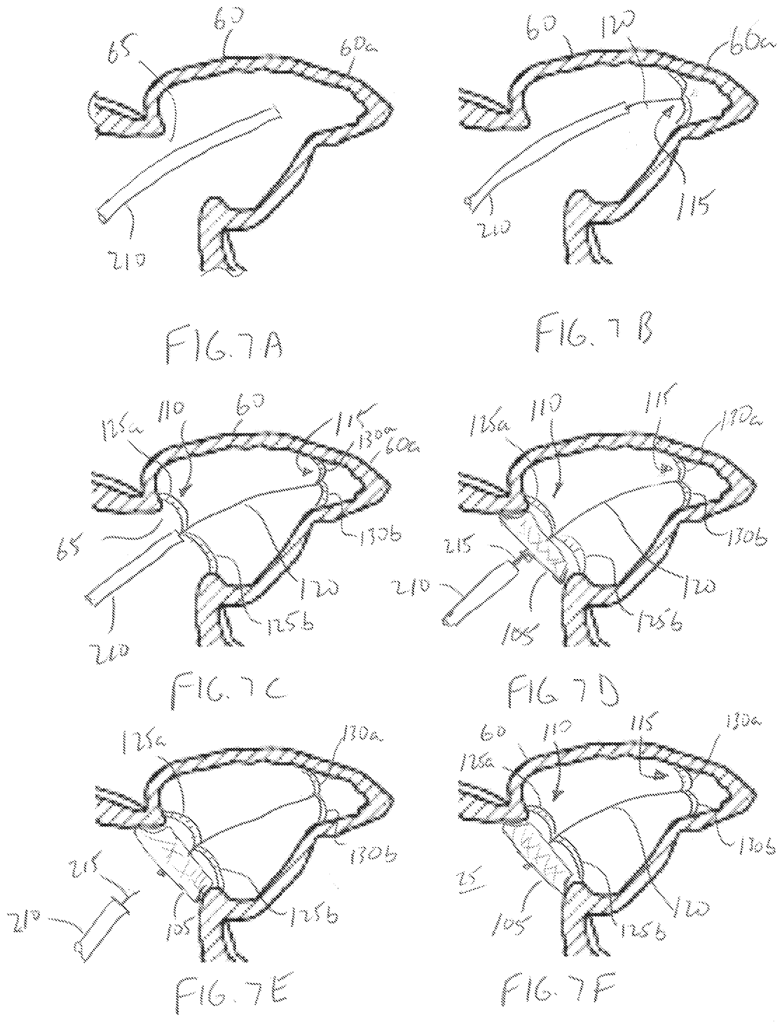

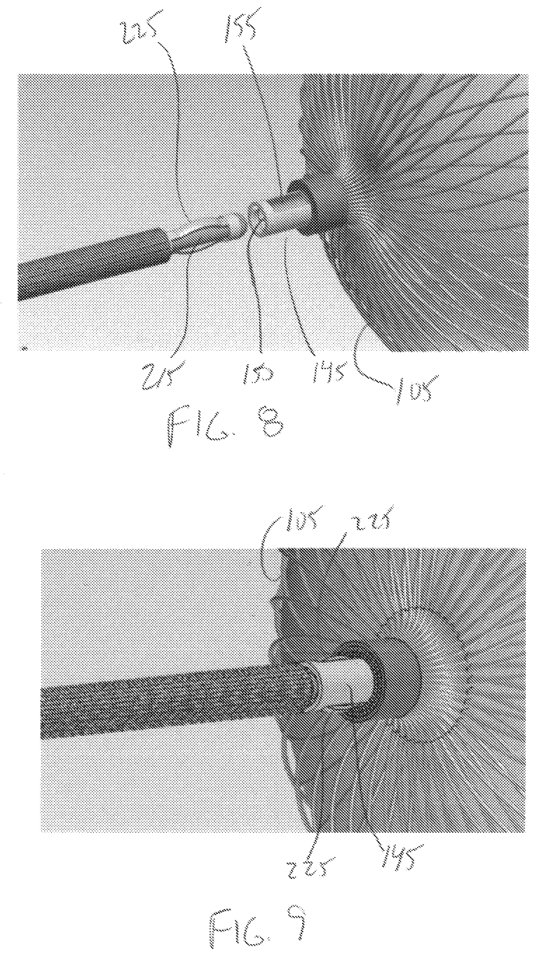

FIGS. 7A-7F show delivery of the implant 100 to the LAA 60. FIG. 7A shows the delivery system 200 positioned near a distal end 60a of the LAA 60 after it is advanced through the heart 10.

FIG. 7B shows initial retraction of the delivery system 200 until the implant 100 begins to exit a distal end and the distal petal anchor 115 is out. Once the distal petal anchor 115 exits the distal end, the asymmetrical petals 130a, 130b self-expand to their original shape and contact the inner wall of the LAA to distally anchor the implant 100.

FIG. 7C shows continued retraction of the delivery system 200 until the proximal petal anchor 110 is out of the distal end. Once the proximal petal anchor 110 exits the distal end, the asymmetrical petals 125a, 125b self-expand to their original shape and contact the inner wall of the LAA to proximally anchor the implant 100.

FIG. 6D shows further retraction of the delivery system 200 until the expandable braided disk 105 is out of the distal end and positioned proximate the LAA ostium 65 of the LAA 60. Once the expandable braided disk 105 exits the distal end it begins to self-expand.

FIG. 7E shows expandable braided disk 105 fully expanded within the LAA ostium 65 to seal the LAA 60. The delivery system 200 is disconnected from the implant 100.

FIG. 7F shows the implant 100 in place, with the proximal and distal petal anchors 110, 115 fully expanded within the ostium 60 and the braided disk 105 fully expanded within the LAA ostium 65 and fluidly sealing the LAA 60 from the left atrium 25.

FIG. 8 is a perspective view showing the components of the connect/disconnect feature that are configured to couple the shaft 215 of a delivery system 200 with the shaft 120 of the implant 100 for delivery. The coupling of the shafts 120, 215 allow the delivery system 200 to torque implant 100 and have zero release force for the implant 100 when the components are uncoupled. This provides the ability to torque the asymmetrical petals of the implant for optimum seal and optimum durability.

The proximal end of the implant 100 includes a coupler 145 having a central opening 150 and slots 155. The distal end of the shaft 215 includes one or more engagement arms 145 having springlike properties that allow them to deflect and spring back to the original position.

FIG. 9 is perspective view showing the implant 100 coupled to the delivery system 200. The distal end of the shaft 215 is sized for insertion into the central opening 150. During insertion, a curved distal portion of the engagement arms/springs 225 contacts the coupler 145 and deflects inwardly into the central opening 150 until the engagement arms/springs 225 line up with the slots 155. Then the spring arms 225 return to their original shape and engage the slots 155. When the engagement arms/springs 225 are coupled with the slots 155, the delivery system 200 may rotate or torque the implant 100 in the LAA 60.

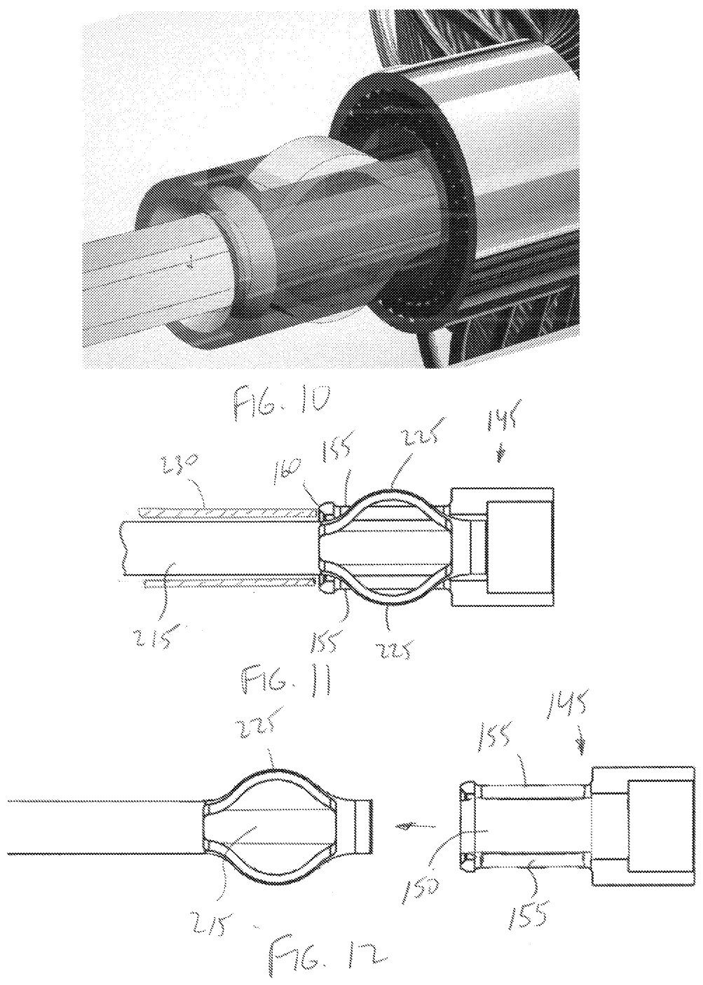

FIG. 10 is perspective view of the connect/disconnect feature of the invention showing the delivery system 200 coupled to the implant 100. The connect/disconnect feature shows the distal end of the Inner Steerable Sheath 210 and engagement arms 225 within the central opening 150 of the coupler 145 with the engagement arms 225 positioned within the slots 155. The coupler 145 is semi-transparent to show more details of the connection.

FIG. 11 is a sectional view showing the engagement of the distal end of the shaft 215 and engagement arms/springs 225 coupled to the coupler 145 and slots 155. To disconnect the delivery system 200 from the implant 100, a removal tube 230 is distally slid over the shaft 215 until it slides over 155 and compressed the engagement arms/springs 225 and stops the proximal end of the coupler 145. Once the engagement arms/springs 225 are “sheathed”, the shaft 215 is advanced distally, to aid in straightening the engagement arms/spring. Both of these actions cause the engagement arms/springs 225 to deflect inward, allowing removal of the outer shaft from the central opening of the coupler 145.

FIG. 12 is a sectional view showing the delivery system 200 disengaged from the implant 10.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

1. An implant for sealing the left atrial appendage (LAA) from the left atrium, comprising:
a body having a proximal end and a distal end;
a disk coupled to the body near the proximal end, the disk configured to seal the LAA from the left atrium;
a proximal anchor having proximal asymmetrical proximal petals comprising two or more size pedals coupled to the body near the proximal end, the proximal asymmetrical petals having a rearward curvature configured to engage a wall of the LAA near a proximal end; and
a distal anchor having distal asymmetrical distal petals comprising two or more size pedals coupled to the body on opposing sides near the distal end, the distal asymmetrical petals having a rearward curvature configured to engage a wall of the LAA near a distal end of the LAA.

2. The implant of claim 1, wherein the disk has multiple/adjustable diameters to seal the LAA ostium.

3. The implant of claim 2, wherein the multiple/adjustable diameters include:
a proximal diameter configured to engage a wall area around a proximal side of the LAA ostium, and
a distal diameter configured to fit within the LAA ostium to keep the disk centered in the LAA ostium.

4. The implant of claim 1, wherein the disk is self-expanding.

5. The implant of claim 4, wherein the self-expanding disk is configured to collapse or compress into a delivery configuration for delivery of the implant to the LAA, and then self-expand after delivery.

6. The implant of claim 1, wherein the proximal asymmetrical petals engage the wall of the LAA on a distal side of the LAA ostium and include an anchor engagement force configured to hold the disk against the wall area.

7. The implant of claim 1, wherein the asymmetrical pedals are curved pedals (like fish hook).

8. The implant of claim 1, wherein the asymmetrical pedals include multiple groups of pedals on opposite sides of the body.

9. The implant of claim 8, wherein the groups of asymmetrical pedals include two or more petals per group.

10. The implant of claim 1, wherein the asymmetrical pedals are self-expanding.

11. The implant of claim 1, wherein the self-expanding disk is configured to collapse or compress into a delivery configuration for delivery of the implant to the LAA, and then self-expand after delivery.

12. The implant of claim 1, wherein the asymmetrical pedals are adjustable in size.

13. The implant of claim 12, wherein the asymmetrical pedals are adjustable either individually or in groups.

14. An implant for sealing the left atrial appendage (LAA) from the left atrium, comprising:
a body having a proximal end and a distal end;
an expandable disk coupled to the body near the proximal end, the disk includes two diameters to seal the LAA from the left atrium:
a proximal diameter configured to engage a wall area around a proximal side of the LAA ostium, and
a distal diameter configured to fit within the LAA ostium to keep the disk centered in the LAA ostium;
a proximal anchor coupled to the body near the proximal end configured to engage a wall of the LAA near a distal side of the LAA ostium with enough force to resist pull-out; and
an expandable distal anchor coupled to the body near the distal end, the expandable distal anchor includes distal petals having a rearward curvature configured to engage a wall of the LAA near a distal end with enough force to resist pull-out.

15. The implantable device of claim 14, wherein:
the proximal petals include asymmetrical proximal petals of two different size pedals coupled to the body on opposing sides near the proximal end, and
the distal petals include asymmetrical distal petals of two different size pedals coupled to the body on opposing sides near the distal end.

16. The implantable device of claim 15, wherein the asymmetrical proximal and the asymmetrical distal petals are configured to accommodate the anatomy of the LAA or placement requirement of the implant within the LAA.

17. The implant of claim 14, wherein the proximal anchor and distal anchor are configured to be collapsed or compressed in a delivery configuration for delivery of the implant to the LAA, and then self-expand after delivery.

18. An implant for sealing the left atrial appendage (LAA) from the left atrium, comprising:
a body having a proximal end and a distal end;
an expandable disk coupled to the body near the proximal end, the disk includes two diameters to seal the LAA from the left atrium:
a proximal diameter configured to engage a wall area around a proximal side of the LAA ostium, and
a distal diameter configured to fit within the LAA ostium to keep the disk centered in the LAA ostium;
an expandable proximal anchor having proximal asymmetrical proximal petals of two different size pedals coupled to the body on opposing sides near the proximal end, the proximal asymmetrical petals having a rearward curvature configured to engage a wall of the LAA near a proximal end of the LAA with enough force to resist pull-out; and
an expandable distal anchor having distal asymmetrical distal petals of two different size pedals coupled to the body on opposing sides near the distal end, the distal asymmetrical petals having a rearward curvature configured to engage a wall of the LAA near a distal end of the LAA with enough force to resist pull-out.

19. The implantable device of claim 18, wherein the asymmetrical proximal and the asymmetrical distal petals are configured to accommodate the anatomy of the LAA or placement requirement of the implant within the LAA.

20. The implant of claim 18, wherein the disk, proximal anchor and distal anchor are configured to be collapsed or compressed in a delivery configuration for delivery of the implant to the LAA, and then self-expand after delivery.

21. A connect/disconnect feature between an implant and an implant delivery system, comprising:
a coupler on a proximal end of an implant, the coupler having a cylindrical body with a central opening and one or more slots in the cylindrical body; and
a shaft on a distal end of the implant delivery system having one or more outward extending engagement arms with springlike properties, the shaft being sized for insertion into the central opening and the one or more engagement arms being configured to deflect inwardly from an original shape during insertion of the shaft in the central opening and return to the original shape within the one or more slots to couple the implant to the implant delivery system;
wherein when the one or more engagement arms are positioned within the one or more slots, the implant delivery system is configured to rotate or torque the implant.

22. The connect/disconnect feature of claim 21, further comprising a removal tube configured to slide distally over the shaft to engage the coupler, once engaged, the shaft may be pulled proximately and the one or more engagement arms deflect inwardly from the slots to allow removal of the shaft from the coupler.
